(19) United States
(12) Patent Application Publication
(10) Pub. No.: US 2025/0213773 A1
(43) Pub. Date: Jul. 3, 2025

(54) METHOD AND SYSTEM FOR MONITORING A WASTE STREAM TO INCREASE EFFICIENCY OF A SURGICAL IRRIGATION PROCEDURE

(71) Applicant: CareFusion 2200, Inc., San Diego, CA (US)

(72) Inventors: Christopher MCGINLEY, San Diego, CA (US); Sunil ANAMANDLA, San Diego, CA (US); Joseph J. DAJCS, San Diego, CA (US)

(21) Appl. No.: 18/853,083

(22) PCT Filed: Mar. 29, 2023
(86) PCT No.: PCT/US2023/065090

§ 371 Date: Sep. 30, 2024

Related U.S. Application Data
(60) Provisional application No. 63/325,033, filed on Mar. 29, 2022.

(51) Int. Cl. A61M 1/00; G01N 21/17; G01N 21/33; G01N 21/47; G01N 21/64

(52) U.S. Cl. CPC A61M 1/77; G01N 21/33; G01N 21/47; G01N 21/6486; A61M 2205/3313; G01N 2021/174; G01N 2021/1744; G01N 2021/4711

(57) ABSTRACT

Methods and systems for performing lavage. The method may include providing an irrigation fluid to a surgical area. The method may also include removing via at least one suction line the irrigation fluid and medical waste at the surgical area to generate a fluid stream in the at least one suction line. The method may also include analyzing the fluid stream based on spectroscopy to generate a result. The method may also include generating a feedback signal based on the result. The method may also include when the feedback signal indicates a negative result, lavage is complete and the surgical area is closed, and when the feedback signal indicates a positive result, lavage is not complete and the method of performing lavage is repeated.

### TECHNICAL FIELD

Aspects of the present disclosure are directed to methods and systems for monitoring a waste stream to increase efficiency of a surgical irrigation procedure. Specifically, aspects of the present disclosure are directed to using spectroscopy to determine the presence of a target material in the waste stream thereby increasing the efficiency of the surgical irrigation procedure.

### BACKGROUND

Irrigation of a surgical cavity, otherwise used interchangeably herein as lavage, is conducted during most surgical procedures in an effort to minimize the presence of debris and/or microorganisms that could adversely impact the outcome of a patient. The ability to minimize debris and/or microorganisms can help reduce and/or eliminate complications, such as infections, that can be catastrophic to the recovery of a patient.

Despite the importance of the irrigation process, there is no standard practice for a surgeon to distinguish when the surgical cavity has been sufficiently flushed and the wound is ready to be closed. A surgeon is left to merely rely on experience and the appearance for the surgical cavity to determine when the irrigation process is complete. These practices are highly variable and thereby reduce the overall effectiveness of the irrigation process.

For example, conventional techniques require a surgeon to visually assess and rely on experience to know when sufficient irrigation has been performed. This practice is highly variable based on the following factors: 1) relying on having a clear line of site to the surgical cavity; 2) limited sensitivity based on use of visual inspection; 3) variable from surgeon-to-surgeon based on the non-specific or quantifiable requirement to “know” the desired appearance for a clean surgical cavity.

There is thus a need in the art for providing the surgeon an indication when the surgical cavity has been efficiently cleaned and when continued flushing is no longer resulting in the removal of debris and/or microorganisms. More specifically, there is a need in the art for a standard that surgeons can rely on to determine when irrigation is complete and the wound may be closed.

### SUMMARY

The following presents a simplified summary of one or more aspects of the disclosure in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects of the disclosure in a simplified form as a prelude to the more detailed description that is presented later.

According to some aspects, the present disclosure is directed to methods and systems for monitoring a waste stream in a surgical procedure. The method includes providing an irrigation fluid to a surgical area. The method may also include removing via a suction line the irrigation fluid and medical waste at the surgical area to generate a fluid stream in the suction line. The method may also monitoring the fluid stream based on spectroscopy to generate a result. The method may also include providing feedback to a user based on the result.

According to some aspects, the present disclosure is directed to methods and systems for performing lavage. The method includes providing an irrigation fluid to a surgical area. The method also includes removing via at least one suction line the irrigation fluid and medical waste at the surgical area to generate a fluid stream in the suction line. The method also includes analyzing the fluid stream to generate a result. The method also includes generating a feedback signal based on the result. The method also includes when the feedback signal indicates a negative result, lavage is complete and the surgical area is closed, and when the feedback signal indicates a positive result, lavage is not complete and the method of performing lavage is repeated.

To the accomplishment of the foregoing and related ends, the one or more aspects of the disclosure comprise the features hereinafter fully described and particularly pointed out in the claims. The following description and the annexed drawings set forth in detail certain illustrative features of the one or more aspects. These features are indicative, however, of but a few of the various ways in which the principles of various aspects can be employed, and this description is intended to include all such aspects and their equivalents.

### BRIEF DESCRIPTION OF THE DRAWINGS

The novel features believed to be characteristic of aspects described herein are set forth in the appended claims. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of illustrative embodiments when read in conjunction with the accompanying drawings, wherein:

FIG. 1 illustrates a portion of an example system according to aspects of the present disclosure;

FIG. 2 illustrates another portion of the example system according to aspects of the present disclosure;

FIG. 3 illustrates an example suction monitoring assembly according to aspects of the present disclosure;

FIGS. 4A, 4B and 4C illustrates an example device for measuring an amount of material in the irrigation system according to aspects of the present disclosure;

FIG. 5 is a flowchart illustrating an example method for monitoring a waste stream according to aspects of the present disclosure;

FIG. 6 presents an example system diagram of various hardware components and other features, for use in accordance with aspects of the present disclosure; and

FIG. 7 is a block diagram of various example system components, for use in accordance with aspects of the present disclosure

### DETAILED DESCRIPTION

The following includes definitions of selected terms employed herein. The definitions include various examples and/or forms of components that fall within the scope of a term and that can be used for implementation. The examples are not intended to be limiting.

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well known structures and components are shown in block diagram form in order to avoid obscuring such concepts.

Several aspects of certain systems will now be presented with reference to various example systems and methods. These systems and methods will be described in the following detailed description and illustrated in the accompanying drawings by various blocks, modules, components, circuits, steps, processes, algorithms, etc. (collectively referred to as “elements”). These elements can be implemented using electronic hardware, computer software, or any combination thereof. Whether such elements are implemented as hardware or software depends upon the particular application and design constraints imposed on the overall system.

As used herein, the term “lavage fluid” refers to a fluid suitable for a lavage process as described herein. As used herein, “lavage” refers to the irrigation of a body cavity, a surgical cavity, and/or an external wound.

According to some aspects, the lavage fluid may comprise an antiseptic solution. As used herein, an “antiseptic solution” refers to a solution comprising at least a solvent and one or more antiseptic agents. According to some aspects, the antiseptic solution comprises an aqueous solution. As used herein, the term “aqueous solution” refers to a solution wherein the solvent comprises at least a majority of water. It should be understood that in some examples, the solvent may comprise or consist of water. According to some aspects, the antiseptic solution comprises an alcoholic solution. As used herein, the term “alcoholic solution” refers to a solution wherein the solvent comprises at least a majority of alcohol. It should be understood that in some examples, the solvent may comprise or consist of one or more alcohols. Non-limiting examples of alcohols include, but are not limited to, ethanol, isopropyl alcohol, n-propanol, and combinations thereof.

In one non-limiting example, the antiseptic agent may comprise a cationic molecule (i.e., a molecule having a positive charge), such as a cationic surfactant or a cationic biguanide derivative (i.e., a compound derived from biguanide). According to some aspects, the antiseptic agent may comprise a bis-(dihydropyridinyl)-decane derivative (i.e., a compound derived from bis-(dihydropyridinyl)-decane). According to some aspects, the antiseptic agent may comprise an octenidine salt and/or a chlorhexidine salt. According to some aspects, the antiseptic agent may comprise alexidine, octenidine dihydrochloride, chlorhexidine gluconate, or a combination thereof.

Additionally or alternatively, the antiseptic agent may comprise iodine. According to some aspects, the iodine may be provided as an iodine complex, such as povidone-iodine (PVPI), nonylphenoxy-(ethyleneoxy)-iodine, polyethylene oxy polyprop leneoxy-iodine, undecoylinium-chloride-iodine, iodine povacrylex, and combinations thereof.

Additionally or alternatively, the antiseptic agent may comprise an oxidant (i.e., an oxidizing agent). Non-limiting examples of oxidants according to the present disclosure include, but are not limited to, sodium hypochlorite, hydrogen peroxide, and combinations thereof.

Additionally or alternatively, the antiseptic agent may comprise antibiotics. According to some aspects, the antibiotics may be bacitracin, vancomycin, gentamycin, ancef, clindamycin and polymixin, and combinations thereof.

Additionally or alternatively, the antiseptic agent may comprise Bactisure and XPerience. According to some aspects, the Bactisure and XPerience may be (1) sodium citrate (˜30 g/L), citric acid (˜32 g/L) and sodium lauryl sulfate (˜1g/L), and (2) sodium acetate (˜30 g/L), acetic acid (˜50 g/L) and benzalkonium chloride (˜1 g/L) and ethanol (˜100 g/L), and (3) combinations thereof.

The antiseptic agent may have an antimicrobial activity sufficient to provide an acceptable log reduction of microbes in a certain time period. It should be understood that as used herein, the term “microbes” may refer to any microorganism to be killed and/or removed as a result of lavage. Example microbes include bacteria, fungi, viruses, and combinations thereof.

Example bacteria include drug-resistant and drug-sensitive, but are not limited to, Streptococcus spp. (e.g., S. mutans, S. pyogenes, S. salivarius, S. sanguis), Staphylococcus spp. (e.g., S. aureus, S. epidermidis, S. haemolyticus, S. hominis, S. simulans, S. saprophyticus), Enterococcus spp. (e.g., E. faecalis E. faecium, and E. hirae), Bacteroides fragilis, Cutibacterium acnes (formerly Propionibacterium acnes), Clostridium difficile (spore and vegetative cells), Pseudomonas aeruginosa, Escherichia coli, Burkholderia cepacia, Proteus mirabilis, Klebsiella spp. (e.g., K. aerogenes, K. pneumoniae), Acinetobacter baumannii,. Micrococus luteus, Haemophilus influenza, and Serratia marcescens.

Example fungi include drug-resistant and drug-sensitive, but are not limited to, Aspergillus brasiliensis, Candida spp. (C. albicans, C. aurus, C. dubliniensis, C. glabrata, C. guillermondii, C. kefyr (formerly C. pseudotropicalis), C. krusei, C. lusitaniae, C. tropicalis), Epidermophyton floccosum, Microsporum spp (e.g., M. gypseum, M. canis), and Trichophyton mentagrophytes.

Example viruses include, but are not limited to, DNA and RNA genomes that are single stranded or double stranded, have sense or antisense orientation, protein coat (capsid) with or without a lipid envelope, such as, cytomegalovirus (CMV), human immunodeficiency virus (HIV), herpes simplex virus types 1 (HSV-1) and 2 (HSV-2), influenza virus, parainfluenza virus, norovirus, and coronavirus.

Example bacteria include, but are not limited to, Streptococcus, Staphylococcus, enterococcus, pseudomonas, Streptococcus mutans, S. pyogenes (group A ß-hemolytic streptococci), S. salivarius, S. sanguis, Staphylococcus aureus S. epidermidis, S. haemolyticus, S. hominis, S. simulans, S. saprophyticus, methicillin/oxacillin-resistant (MRSA/ORSA) and methicillin/oxacillin-susceptible Staphylococci (MSSA/OSSA), Enterococcus (e.g., E. faecalis E. faecium, and E. hirae), vancomycin-resistant Enterococcus (VRE) and vancomycin-susceptible Enterococcus (VSE), Bacteroides fragilis, Propionibacterium acnes, Propionibacterium, Clostridium difficile (spore and vegetative cells), Selenomonas, Pseudomonas aeruginosa, Escherichia coli, Burkholderia cepacia, Proteus mirabilis, Gardnerella vaginalis, Klebsiella aerogenes, K. pneumoniae, K. pneumoniae multidrug resistant (MDR), Acinetobacter baumannii, A. baumannii MDR, Achromobacter xylosoxidans. Micrococus luteus, Ralstonia pickettii, Haemophilus influenza, and Serratia marcescens.

Example fungi include, but are not limited to, aspergillus, candida, Aspergillus niger, Candida albicans, C. aurus, C. dubliniensis, C. glabrata (formerly Torulopsis glabrata), C. guillermondii, C. kefyr (formerly C. pseudotropicalis), C. krusei, C. lusitaniae, C. tropicalis, Epidermophyton floccosum, Microsporum gypseum, M. canis, and Trichophyton mentagrophytes.

Example viruses include, but are not limited to, those having a lipid component in their outer coat or have an outer envelope such as cytomegalovirus (CMV), human immunodeficiency virus (HIV), herpes simplex virus types 1 (HSV-1) and 2 (HSV-2), influenza virus, parainfluenza virus, variola virus (smallpox virus), vaccinia, norovirus, and coronavirus.

Example debris includes, but is not limited to, bone fragments, tissue, blood, bile, puss, mucus, stool, cartilidge, fat, urine, environmental contamination (i.e., dirt, non-sterile fluid, hair, etc.).

Example non-endogenous proteins include, but not limited to, stool, gut microbiota, proteins from microorganism and fungi.

According to some aspects, the certain time period may be a period of no more than about five minutes, optionally no more than about four minutes, optionally no more than about three minutes, optionally no more than about two minutes, and optionally no more than about one minute.

According to some aspects, the certain time period may be no more than about 120 seconds, optionally no more than about 105 seconds, optionally no more than about 90 second, optionally no more than about 75 seconds, optionally no more than about 60 seconds, optionally no more than about 45 seconds, optionally no more than about 30 seconds, and optionally no more than about 15 seconds.

It should be understood that “an acceptable log reduction” may be microbe-dependent. For example, an acceptable log reduction as described herein may refer to an acceptable log reduction of one type of microbe present on a surface (e.g., present in a body cavity or at an external wound site), a combination of two more types of microbes present on a surface, or total microbes present on a surface.

According to some aspects, an acceptable log reduction may be at least about 1.0, optionally at least about 1.1, optionally at least about 1.2, optionally at least about 1.3, optionally at least about 1.4, optionally at least about 1.5, optionally at least about 1.6, optionally at least about 1.7, optionally at least about 1.8, optionally at least about 1.9, optionally at least about 2.0, optionally at least about 2.1, optionally at least about 2.2, optionally at least about 2.3, optionally at least about 2.4, optionally at least about 2.5, optionally at least about 2.6, optionally at least about 2.7, optionally at least about 2.8, optionally at least about 2.9, optionally at least about 3.0, optionally at least about 3.1, optionally at least about 3.2, optionally at least about 3.3, optionally at least about 3.4, optionally at least about 3.5, optionally at least about 3.6, optionally at least about 3.7, optionally at least about 3.8, optionally at least about 3.9, optionally at least about 4.0, optionally at least about 4.1, optionally at least about 4.2, optionally at least about 4.3, optionally at least about 4.4, optionally at least about 4.5, optionally at least about 4.6, optionally at least about 4.7, optionally at least about 4.8, optionally at least about 4.9, and optionally at least about 5.0.

According to some aspects, the antiseptic agent may be present in the antiseptic solution in a concentration sufficient to provide an acceptable log reduction of microbes in a certain time period as described herein. According to some aspects, the antiseptic agent may be present in the antiseptic solution at a concentration of between about 0.001 and 5% w/v, optionally between about 0.001 and 2.5% w/v, optionally between about 0.001 and 1% w/v, optionally between about 0.001 and 0.1% w/v, optionally between about 0.001 and 0.01% w/v, optionally between about 0.01 and 5% w/v, optionally between about 0.01 and 2.5% w/v, optionally between about 0.01 and 2% w/v, optionally between about 0.01 and 1.5% w/v, optionally between about 0.01 and 1% w/v, and optionally about 0.5% w/v.

According to some aspects, the antiseptic agent may be present in the antiseptic solution at a concentration of between about 0.1 and 0.9% w/v, optionally between about 0.2 and 0.8% w/v, optionally between about 0.3 and 0.7% w/v, and optionally between about 0.4 and 0.6% w/v.

According to some aspects, the antiseptic agent may be present in the antiseptic solution at a concentration of between about 0.1 and 10% w/v, optionally between about 0.2 and 1% w/v, optionally between about 0.3 and 1% w/v, and optionally between about 0.4 and 1% w/v.

It should be understood that according to some aspects, the lavage fluid is not necessarily an antiseptic solution as described herein and may be any medically acceptable fluid configured to perform a lavage process as described herein. In one non-limiting example, the lavage fluid may comprise a saline solution. The saline solution may comprise water and sodium chloride in a medically acceptable concentration, such as between about 0.1 and 1%, w/v, optionally about 0.45% w/v, and optionally about 0.9% w/v.

According to some aspects, the lavage fluid may be the lavage fluid as described in U.S. application Ser. No. 17/152,565, hereby incorporated by reference in its entirety.

According to some aspects, the lavage fluid may also contain radioisotopes, in other words, a radioactive isotope, for example, fluorine-18, gallium-67, krypton-81m, rubidium-82, nitrogen-13, technetium-99m, indium-111, iodine-123, xenon-133, and thallium-201.

The device, as described below, according to the present disclosure comprises a body configured to contain a lavage fluid as described herein. It should be understood that as used herein, “dispense” (alternatively referred to as “discharge”) may refer to transferring the lavage fluid to an application member in fluid communication with the body and/or it may refer to transferring the lavage fluid from an application member to a surface.

According to some aspects, the body may comprise a body material that is compatible with the lavage fluid contained therein, that is, a material that does not chemically or physically react with the lavage fluid or otherwise render the lavage fluid unfit for medical use.

According to an aspect of the disclosure, the fluid lines may comprise a semi-

flexible conduit, a flexible conduit, or a rigid conduit. Further, the fluid lines may be transparent and able to be visually seen through.

Accordingly to an aspect, the disclosure does not require a line of sight in order to determine when a suitable amount of irrigation of the surgical cavity has been completed. By monitoring the irrigation solution waste stream, this disclosure would be able to detect the presence of microorganisms and/or debris being removed from the surgical cavity at any location that contacts the irrigation solution. This eliminates the restriction of only being able to assess those areas that are visual, and also greatly facilitates monitoring of debris and/or microorganisms for other organs or tissue that may not be visually present.

Accordingly to another aspect, the disclosure has a significantly high sensitivity compared to conventional practices that rely on visual inspection. While the conventional practices rely on human vision (and thus may not be sufficient to detect material that is not discernible to the human eye (i.e., sub-micron particles)), the disclosure uses analytical techniques and other detection methodologies that offer significantly higher sensitivity compared to the human eye. The disclosure eliminates the limited sensitivity for the assessment and thereby greatly increases the effectiveness for the assessment when a surgical cavity has been sufficiently irrigated.

Further, accordingly to another aspect, the disclosure does not require the use of the subjective methodology from conventional practices, and instead has a quantifiable and non-subjective methodology to assess the irrigation of the surgical cavity. By utilizing a non-subjective methodology, the disclosure eliminates the restrictions associated with surgeon-to-surgeon and patient-to-patient variability that hinder the conventional practices. Another advantage is that the non-subjective methodology does not require any specific training and/or experience by the surgeon, thereby further enhancing the reproducibility in determining when the surgical cavity has been sufficiently irrigated.

The systems and methods, as described below, may monitor fluid lines for material, for example, microbes, debris, non-endogenous proteins, and/or other aspects.

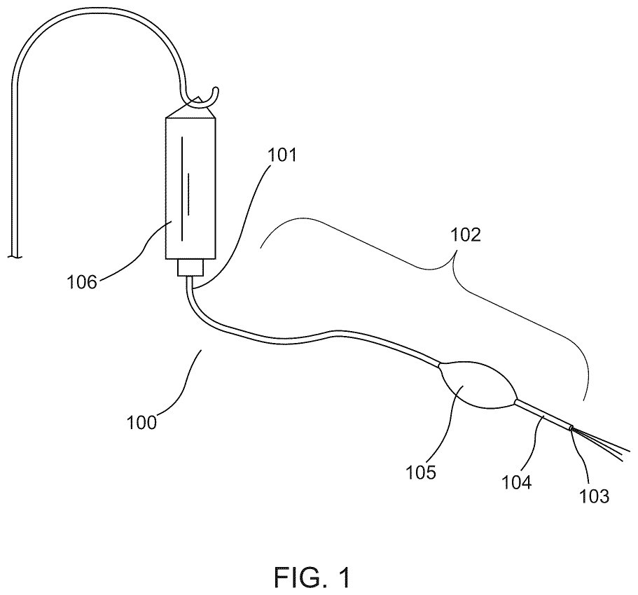

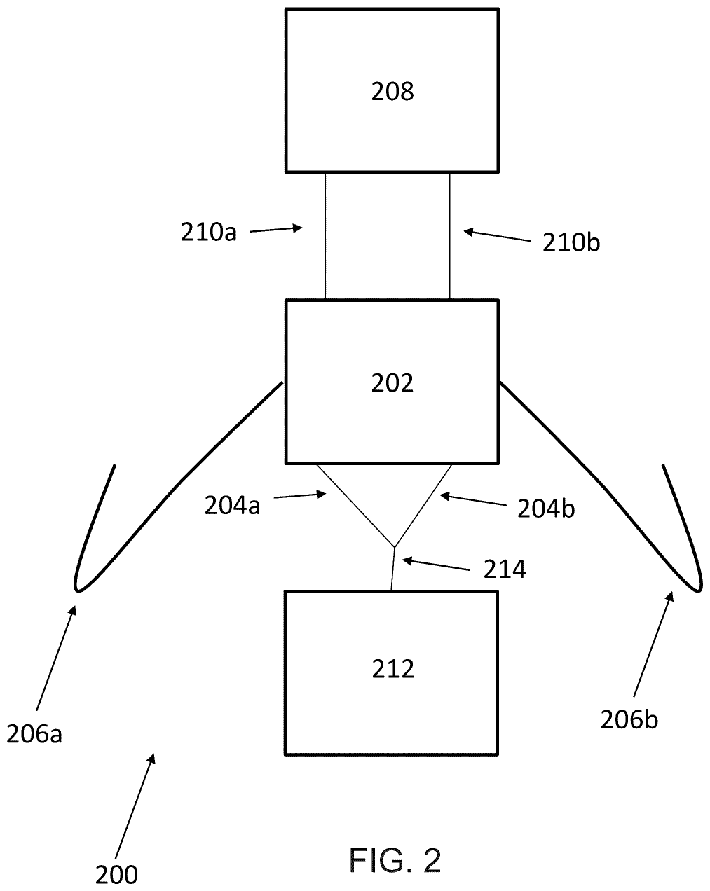

FIGS. 1 and 2 (as described below) together make up a preferred irrigation system. FIG. 1 illustrates an example portion of an irrigation system for use in a surgical procedure in accordance with one aspect of the disclosure. As illustrated in FIG. 1, the application member 100 may comprise a connection portion 101 and a discharge portion 102. Connection portion 101 may be configured to connect application member 100 with body 106 as described herein. Discharge portion 102 may comprise one or more discharge apertures 103 configured to dispense a fluid (e.g., an antiseptic solution) onto a surface, such as a surgical site during a lavage process. It should be understood that discharge portion 102 may comprise a conduit 104 that may be a semi-flexible conduit, a flexible conduit, or a rigid conduit.

As illustrated in FIG. 1, application member 100 may further comprise a dispensing aid 105 as described herein, such as a pump. Dispensing aid may be a mechanical pump. Additionally or alternatively, dispensing aid may be a motorized pump.

It should be understood that application member 100 having dispensing aid 105 as described herein may dispense a lavage fluid (e.g., an antiseptic solution) from body 106 upon actuation of dispensing aid 105 (e.g., actuation of a pump as described herein). Additionally or alternatively, dispensing aid 105 may function to dispense fluid from body 106 in conjunction with the force of gravity. For example, FIG. 1 shows an example body 106, that is, a body configured such that at least a portion of the lavage fluid contained therein is dispensed by the force of gravity when provided in a certain orientation. It should be understood that the dispensing aid will advantageously allow a user to control the fluid flow force, the fluid flow rate, and/or the fluid flow pattern (e.g., pulsed or constant) of the dispensed lavage fluid.

While the example shown in FIG. 1 shows discharge portions having one discharge aperture, it should be understood that the discharge portion may comprise two, three, four, or more discharge apertures. Each of the discharge apertures may be the same size as or a different size from one or more of the other discharge apertures. Additionally or alternatively, each of the discharge apertures may have the same shape as or a different shape from one or more of the other discharge apertures. The shape and/or size of the one or more discharge apertures may be selected to provide a certain fluid flow force, fluid flow rate, and/or fluid flow pattern. According to some aspects, the shape and/or size of the one or more discharge apertures may be adjustable such that the fluid flow force, fluid flow rate, and/or fluid flow pattern of a dispensed fluid may be adjustable.

While the example shown in FIG. 1 shows one example of a discharge portion of an irrigation system used in surgical procedures, any conventional discharge portions of an irrigation system may be used. In addition or alternatively, the body 106 may be made of a deformable material, such as plastic, which allows the lavage fluid to be discharged by applying a compression force to the body.

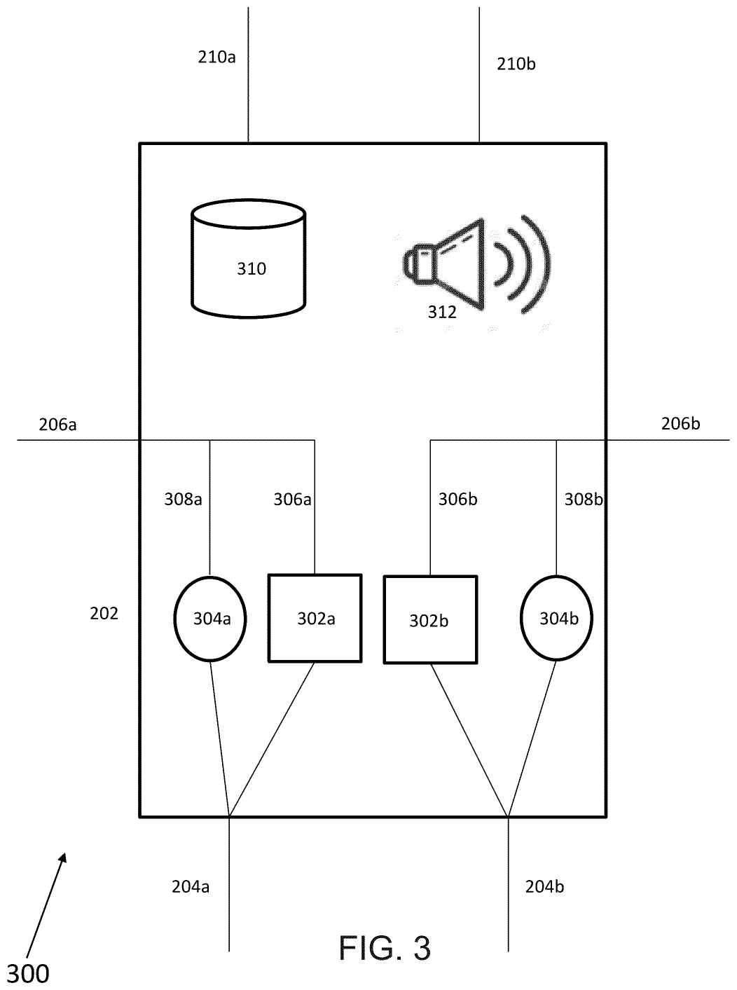

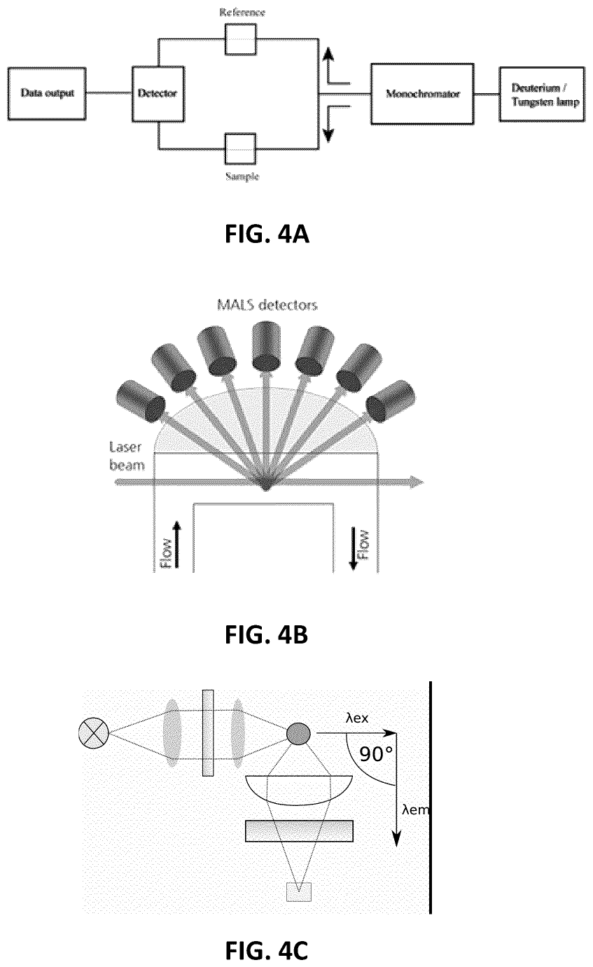

FIG. 2 illustrates an example portion of an irrigation system for use in a surgical procedure in accordance with one aspect of the disclosure. Illustrated in FIG. 2 is a medical suction system 200. As illustrated in FIG. 2, for example, a medical suction system 200 can include a suction monitoring assembly 202. The suction monitoring assembly 202 is described in detail below in accordance with FIGS. 3 and 4. In accordance with an aspect of the disclosure, the suction monitoring assembly 202 and/or components thereof may be disposable. The suction monitoring assembly 202 may include a plurality of lines 204 and 206. In one aspect of the disclosure suction lines 206 are in and lines 204 are out. The lines 204 and 206 can be configured to facilitate fluid communication between fluid lines external to the suction monitoring assembly 202 and fluid lines internal to the suction monitoring assembly 202.

In accordance with an aspect of the disclosure, the medical suction system 200 includes a first suction line 206a. The medical suction system 200 can, in an aspect of the disclosure, include a second suction line 206b. The suction monitoring assembly 202 can be configured to utilize the first and second suction lines 206a, 206b simultaneously and/or in isolation.

In accordance with an aspect of the disclosure, the medical suction system 200 can include a vacuum source 208. In accordance with an aspect of the disclosure, the vacuum source 208 is a hospital vacuum source. In accordance with an aspect of the disclosure, the vacuum source 208 is a pump. Many variations are possible, as would be known to a person skilled in the art. In accordance with an aspect of the disclosure, the vacuum source 208 is fluidly connected to the suction monitoring assembly 202 via a first vacuum line 210a. In accordance with an aspect of the disclosure, a second vacuum line 210b connects the vacuum source 208 to the suction monitoring assembly 202. The medical suction system 200 can be configured to utilize the first and second vacuum lines 210a, 210b simultaneously and/or in isolation.

In accordance with an aspect of the disclosure, the medical suction system 200 includes a waste tank 212. The waste tank 212 can be configured to store medical waste, which may include one or more materials selected from blood, tissue, bone, other bodily fluids and/or other debris, microorganisms, non-endogenous proteins and/or dispensed lavage fluid (i.e., lavage fluid which has been in contact with the surgical area which may or may not contain additional materials based upon the status of the lavage). In accordance with an aspect of the disclosure, the waste tank 212 is fluidly connected to a waste line 214 or other disposal lines. The waste tank 212 can be connected to the suction monitoring assembly 202 via the waste line 214.

The suction monitoring assembly 202 can monitor one or more materials in the medical waste removed from the body cavity, a surgical cavity, and/or an external wound during a surgical procedure. The same reference numbers illustrated in reference to FIG. 3 are used for FIG. 2. In one aspect of the disclosure, the suction monitoring assembly 202 may include a sampling device 302a and 302b which are connected in-line to the corresponding first suction line 206a and second suction line 206b, respectively. The sampling device 302 is described in detail below with reference to FIG. 4. The sampling devices 302a and 302b are connected to fluid lines 306a and 306b, respectively.

Further, in accordance with another aspect, the suction monitoring assembly 202 may include a sampling port 304a and 304b which are connected in-line to the corresponding first suction line 206a and second suction line 206b, respectively. The sampling port 304 is described in detail below with reference to FIG. 4. The sampling ports 304a and 304b are connected to fluid lines 308a and 308b, respectively.

In one aspect of the disclosure a user, for example, a surgeon may be able to determine when the irrigation is complete by monitoring the fluid passing through the first suction line 206a and the second suction line 206b continuously via the sampling device. In another aspect of the disclosure the fluid may be monitored in parallel with the fluid lines 306 and/or 308. The fluid lines 306 and/or 308 may be of different sizes inner diameters in order to allow for a different flow rates. For example, if a smaller path is used for one fluid line, fluid would flow at a lower rate and may help facilitate select measurement techniques, as described below. In another aspect of the disclosure the fluid may be monitored via the sampling port 304 to allow a sample of the fluid to be obtained at specific times and tested remote from the irrigation system.

In accordance with one aspect of the disclosure the fluid is to monitored for specific materials in order to determine if the irrigation and/or lavage is complete. The material to be monitored in the surgical irrigation waste may vary depending on the type of surgery and/or patient risk factor. For example, the material to be monitored may be more generic (i.e., any material that is not in the irrigation solution and/or lavage fluid solution itself) or more specific (i.e., specific material or microbe, as described above) depending on the risk factor associated with the surgical procedure.

In another aspect of the disclosure, the material to be monitored may also be influenced by factors such as trauma vs non-trauma (trauma surgeries may have less control and more variability of the material that could be present in the surgical cavity), risk for the surgery (potential for inadvertently cutting the bowel during surgical procedure or detection of anastomotic leak in colorectal surgery).

In one aspect of the disclosure, the irrigation system may provide feedback to the user indicating that irrigation should continue or that the irrigation is complete. In one aspect of the disclosure, a visual indication, for example a light 310, may be provided to the user indicating if irrigation should continue or is complete. For example, if continuous monitoring is being performed by the irrigation system, the light 310 may provide immediate feedback to the surgeon as to the effectiveness for the irrigation and/or cleaning of the surgical cavity. The intensity and/or color of the light may be linked to the amount of material that is measured, as described below in relation to FIG. 4. For example, the light 310 may be red indicating more irrigation and/or cleaning of the cavity may be necessary based on the sample device 302. This type of real-time output may also provide aid in determining an area of the surgical cavity that may require additional cleaning. For example, if the surgeon is rinsing a specific area of the wound and immediately removing the waste stream, the real time indication could provide guidance if that specific area requires further irrigation.

In accordance with another aspect of the disclosure, the irrigation system may provide an audible indication, for example a speaker 312, to the user. The audible indication thereby does not require the surgeon and/or user to look at a visual indication. In one aspect of the disclosure, the pitch of the sound, volume and/or the frequency of the audible indication may be linked to the amount of material that is measured by the irrigation system.

Referring to FIG. 3, although two suction lines 206 are illustrated, any number of suction lines may be implemented, for example, 1, 2, 3, 4, etc. Further, although both the sampling port 304 and the sampling device 302 are illustrated, any number or variation of sampling ports or sampling devices may be implemented, for example, 1, 2, 3, 4, etc. For example, a single suction line may be implemented which includes a single sampling device 302 and without a sampling port. In another example, two sampling devices 302a may be included on a single suction line 206a. Any combination and/or variation may be implemented based on the sampling procedure and/or measuring procedure described below.

FIG. 4, containing FIGS. 4A, 4B and 4C illustrate example devices for detecting the presence of and/or measuring the amount of material in the irrigation system in accordance with one aspect of the disclosure, specifically via spectroscopy (spectroscopy, spectroscopy test or test are used interchangeably herein). For example, spectroscopy may be conducted via at least one of an ultraviolet measurement unit as illustrated in FIG. 4A, a multiangle light scattering measurement device as illustrated in FIG. 4B, or a fluorescence emission measurement device as illustrated in FIG. 4C. Spectroscopy, as described below, may be implemented to measure the amount of material within the irrigation solution waste stream. In one aspect of the disclosure, the spectroscopy device may be located within the sampling device 302 for continuous monitoring, and providing feedback to the user. In another aspect of the disclosure the spectroscopy device may be located external and remote to the irrigation system, and where the spectroscopy device is provided samples that are acquired via the sampling port 304.

In accordance with one aspect of the disclosure, spectroscopy may be conducted via an ultraviolet measurement device/method, a multiangle light scattering device/method or a fluorescence emissions device/method, and the like. These techniques are known to general technicians in the field. In general spectroscopy is the study of the interaction between matter and electromagnetic radiation as a function of the wavelength or frequency of the radiation. In other words, spectroscopy is the precise study of light s generalized from visible light to all bands of the electromagnetic spectrum.

In one aspect of the disclosure, the selection of the specific methodology for the spectroscopy (e.g., ultraviolet measurement, multiangle light scattering, phosphorescence or fluorescence emissions) and also the specific experiment parameters (i.e., wavelength or excitation) may be selected based on the known or determined attributes of the targeted material within the irrigation solution waste stream that is being monitored. For example, some wavelengths may be more suitable for proteins and/or microorganisms, while another wavelength may be more general and provide an indication for the presence of a general material. For example, the monitoring of fluorescence measurements may be conducted in order to detect tryptophan amino acid, which may be an indicator for the presence of protein(s). If the presence (or if presence above a certain predetermined threshold) is detected via the spectroscopy, the lavage procedure may be repeated or continued until the absence of such presence. In one aspect of the disclosure, the measurement or the type of specific methodology may be adjusted based on the irrigation solution waste stream, the path length, or a modification of other parameters during the measurement itself.

In one aspect of the disclosure the specific methodology for the spectroscopy, specifically an ultraviolet measurement may be conducted by the device/method illustrated in FIG. 4A. Ultraviolet measurement (or Spectrophotometry) is a quantitative technique used to measure how much a chemical substance absorbs light. This is done by measuring the intensity of light that passes through a sample with respect to the intensity of light through a reference sample or blank. As described above, these variables may be selected based on the known or determined attributes of the target material within the irrigation solution waste stream that is being monitored to determine the presence.

In another aspect of the disclosure the specific methodology for the spectroscopy, specifically the multiangle light scattering may be conducted by the device/method illustrated in FIG. 4B. Multiangle light scattering (MALS) describes a technique for measuring the light scattered by a sample into a plurality of angles. It is used for determining both the absolute molar mass and the average size of molecules in solution, by detecting how they scatter light. As described above, these variables may be selected based on the known or determined attributes of the material within the irrigation solution waste stream that is being monitored to determine the presence. In one aspect of the disclosure, multiangle light scattering may not require a molecule that has a UV or Fluorescent signal. Thus, this methodology may be used for some molecules that cannot be detected with UV.

In another aspect of the disclosure, the fluorescence emissions measurement may be conducted by the device/method illustrated in FIG. 4C. Fluorescence emissions (also known as fluorimetry or spectrofluorometry) is a type of electromagnetic spectroscopy that analyzes fluorescence from a sample. It involves using a beam of light, usually ultraviolet light, that excites the electrons in molecules of certain compounds and causes them to emit light; typically, but not necessarily, visible light. A complementary technique is absorption spectroscopy. In the special case of single molecule fluorescence spectroscopy, intensity fluctuations from the emitted light are measured from either single fluorophores, or pairs of fluorophores. As described above, these variables may be selected based on the known or determined attributes of the material within the irrigation solution waste stream that is being monitored to determine the presence. In one aspect of the disclosure, some specific compounds may contain a fluorescence, thus fluorescence emissions measurement may be implemented for these compounds and may offer another option for detection.

In accordance with another aspect of the disclosure, this test methodology may be incorporated for measurement of the irrigation solution waste stream at a specific time (i.e. the spectroscopy may be conducted on the irrigation stream via the sampling device 302). This test could be used at different times during the irrigation process to monitor to progress of the irrigation in removal of debris and/or microorganisms from the surgical cavity.

In addition, the spectroscopy may be assembled to provide a measurement at discrete intervals. Through the use of constant measuring, the irrigation solution could be added to individual spectroscopy devices at discrete time intervals and thereby providing the surgeon with an indication for the presence of the target material throughout the irrigation process. For example, different spectroscopy devices may be placed in different sampling devices 302. In another example, a single spectroscopy device may be located within a single sampling device 302 but designed to have multiple tests performed at different times.

In one aspect of the disclosure, the spectroscopy test may provide a binary outcome, for example, “yes” the presence of targeted material or “no” the lack of presence of the targeted material during lavage. In another aspect of the disclosure, the spectroscopy test may compare results to threshold(s), thereby providing an infinite number of outcomes based upon the type of targeted material being tested. For example, if any presence of a specific targeted material is harmful to a patient during lavage, a binary outcome may be implemented by the system. In another example, if the presence of a specific targeted material is only harmful at a discrete level at a specific location of a patient, different thresholds may be implemented by the system during lavage. In other words, if material X at level 1 is not harmful in the arm of a patient during lavage, but level 2 is harmful, the same spectroscopy test may provide indications of level 1 or level 2 to a user to determine if lavage should continue or is complete. In another aspect of the disclosure, the spectroscopy test may also provide quantification of the targeted material based on the comparison of thresholds. For example, the comparison of thresholds may provide for a range. If the first threshold is met by the spectroscopy test, more than X amount of the targeted material is present. Additionally, if the second threshold is not met by the spectroscopy test, less than Y amount of the targeted material is present, and so on. In other words, the user may determine a range of targeted material present during lavage based on a plurality of thresholds. For example, as described above, more than X but less than Y amount of the targeted material is present during lavage.

In another aspect of the disclosure, the spectroscopy test may provide exact quantitative numbers/values regarding the amount of targeted material present during lavage, as described above.

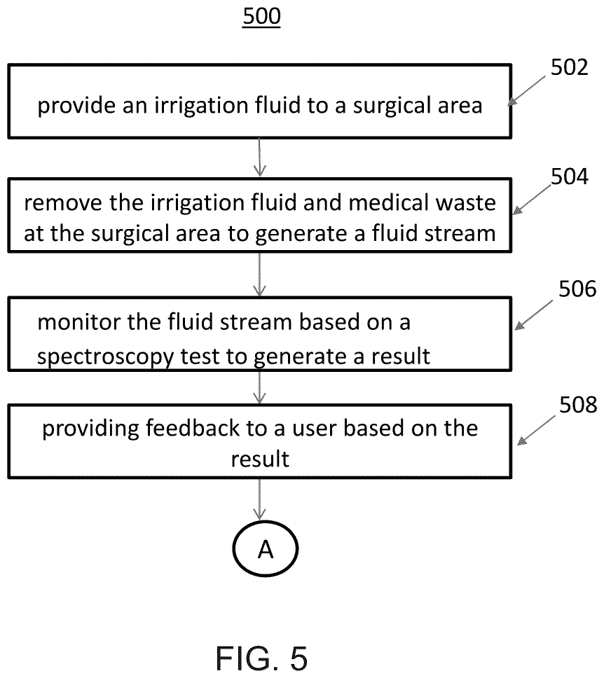

Referring to FIG. 5, therein illustrates a flowchart showing an example method 500 for measuring the amount of material in an irrigation system in accordance with one aspect of the disclosure. For example, method 500 may be performed in conjunction with the irrigation system described above in relation to FIGS. 1-4.

At block 502, the method may include providing an irrigation fluid to a surgical area. As described above, the irrigation fluid may be a lavage fluid. The fluid may be provided to the surgical area via the system described above in relation to FIG. 1.

At block 504, the method may include removing the irrigation fluid and medical waste at the surgical area. These irrigation fluids and medical waste materials mixed together may generate a fluid stream. As described above, the fluid stream is monitored to determine when lavage or irrigation by the surgeon is complete.

At block 506, the method may further include monitoring the fluid stream based on spectroscopy to generate a result. For example, the test may be configured to test for a specific material based on, for example as described above, the type of procedure being performed. In one aspect of the disclosure, the result may be a negative or positive. For example, if the spectroscopy indicates a positive result, this result would indicate that the area would need further lavage. In another example, if the spectroscopy indicates a negative result, this result would indicate that the area is complete of lavage and the wound may be closed.

At block 508, the method may include providing feedback to a user based on the result. For example, the spectroscopy may provide an indication to convey a positive or negative result. In another example, the spectroscopy may trigger an alert, for example as described above, a light or audible noise to convey the result to the user.

At block A, the user would determine if further lavage is needed or the wound may be closed based upon the generated result.

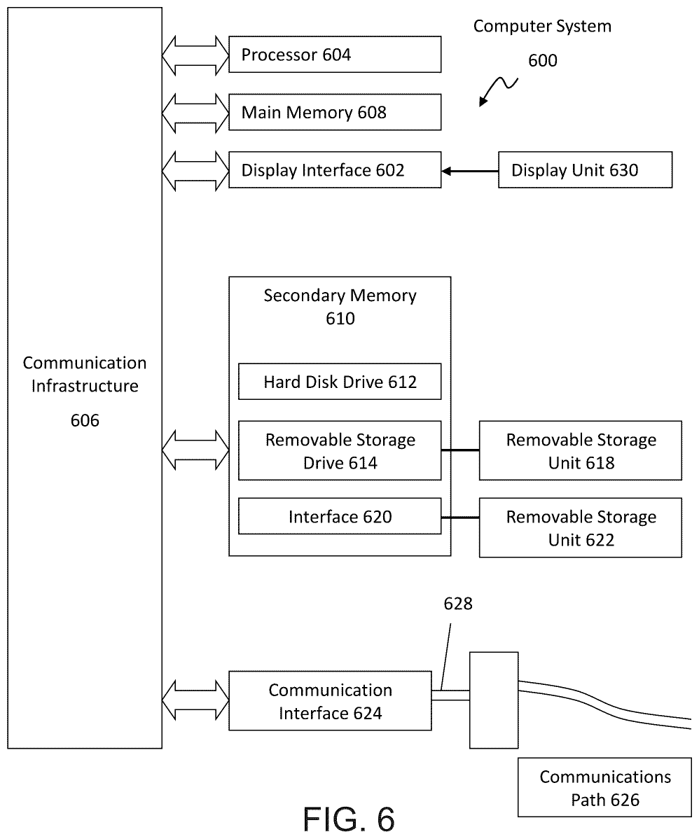

Aspects of the present disclosure may be implemented using hardware, software, or a combination thereof and can be implemented in one or more computer systems or other processing systems. In one aspect, the disclosure is directed toward one or more computer systems capable of carrying out the functionality described herein. An example of such a computer system 600 is shown in FIG. 6.

In one aspect of the disclosure, the suction monitoring assembly 202 may contain a computer system, for example computer system 600, capable of processing and generating the results of the monitoring, as described above. For example, the computer system may generate the result from the outcome of the sampling devices 302a and 302b and/or the sampling ports 304a and 304b. The computer system 600 may further compare the result to a threshold and generate an output signal based on the comparison. The output signal may further be implemented to trigger an alert for the user, as described above. For example, based on the output signal, a light may be illumined and/or a sound may be generated. The user may then either determine the lavage is complete, and close the patient or the lavage is not complete and continue to lavage the patient. In other words, if lavage is not complete for the patient the system alerts the user, and the process may repeat itself until the system determines lavage is complete and alerts the user.

FIG. 6 presents an example system diagram of various hardware components and other features, for use in accordance with an aspect of the present disclosure. Aspects of the present disclosure can be implemented using hardware, software, or a combination thereof and can be implemented in one or more computer systems or other processing systems. In one example variation, aspects described herein can be directed toward one or more computer systems capable of carrying out the functionality described herein. An example of such a computer system 600 is shown in FIG. 6.

Computer system 600 includes one or more processors, such as processor 604. The processor 604 is connected to a communication infrastructure 606 (e.g., a communications bus, cross-over bar, or network). In one example, irrigation system can include processor 604. Various software aspects are described in terms of this example computer system. After reading this description, it will become apparent to a person skilled in the relevant art(s) how to implement aspects described herein using other computer systems and/or architectures.

Computer system 600 can include a display interface 602 that forwards graphics, text, and other data from the communication infrastructure 606 (or from a frame buffer not shown) for display on a display unit 630. Computer system 600 also includes a main memory 608, preferably random access memory (RAM), and can also include a secondary memory 610. The secondary memory 610 can include, for example, a hard disk drive 612 and/or a removable storage drive 614, representing a floppy disk drive, a magnetic tape drive, an optical disk drive, etc. The removable storage drive 614 reads from and/or writes to a removable storage unit 618 in a well-known manner. Removable storage unit 618, represents a floppy disk, magnetic tape, optical disk, etc., which is read by and written to removable storage drive 614. As will be appreciated, the removable storage unit 618 includes a computer usable storage medium having stored therein computer software and/or data.

In alternative aspects, secondary memory 610 can include other similar devices for allowing computer programs or other instructions to be loaded into computer system 600. Such devices can include, for example, a removable storage unit 622 and an interface 620. Examples of such can include a program cartridge and cartridge interface (such as that found in video game devices), a removable memory chip (such as an erasable programmable read only memory (EPROM), or programmable read only memory (PROM)) and associated socket, and other removable storage units 622 and interfaces 620, which allow software and data to be transferred from the removable storage unit 622 to computer system 600.

Computer system 600 can also include a communications interface 624. Communications interface 624 allows software and data to be transferred between computer system 600 and external devices. Examples of communications interface 624 can include a modem, a network interface (such as an Ethernet card), a communications port, a Personal Computer Memory Card International Association (PCMCIA) slot and card, etc. Software and data transferred via communications interface 624 are in the form of signals 628, which can be electronic, electromagnetic, optical or other signals capable of being received by communications interface 624. These signals 628 are provided to communications interface 624 via a communications path (e.g., channel) 626. This path 626 carries signals 628 and can be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 680, a hard disk installed in hard disk drive 670, and signals 628. These computer program products provide software to the computer system 600. Aspects described herein can be directed to such computer program products.

Computer programs (also referred to as computer control logic) are stored in main memory 608 and/or secondary memory 610. Computer programs can also be received via communications interface 624. Such computer programs, when executed, enable the computer system 600 to perform various features in accordance with aspects described herein. In particular, the computer programs, when executed, enable the processor 604 to perform such features. Accordingly, such computer programs represent controllers of the computer system 600.

In variations where aspects described herein are implemented using software, the software can be stored in a computer program product and loaded into computer system 600 using removable storage drive 614, hard disk drive 612, or communications interface 620. The control logic (software), when executed by the processor 604, causes the processor 604 to perform the functions in accordance with aspects described herein as described herein. In another variation, aspects are implemented primarily in hardware using, for example, hardware components, such as application specific integrated circuits (ASICs). Implementation of the hardware state machine so as to perform the functions described herein will be apparent to persons skilled in the relevant art(s).

In yet another example variation, aspects described herein are implemented using a combination of both hardware and software.

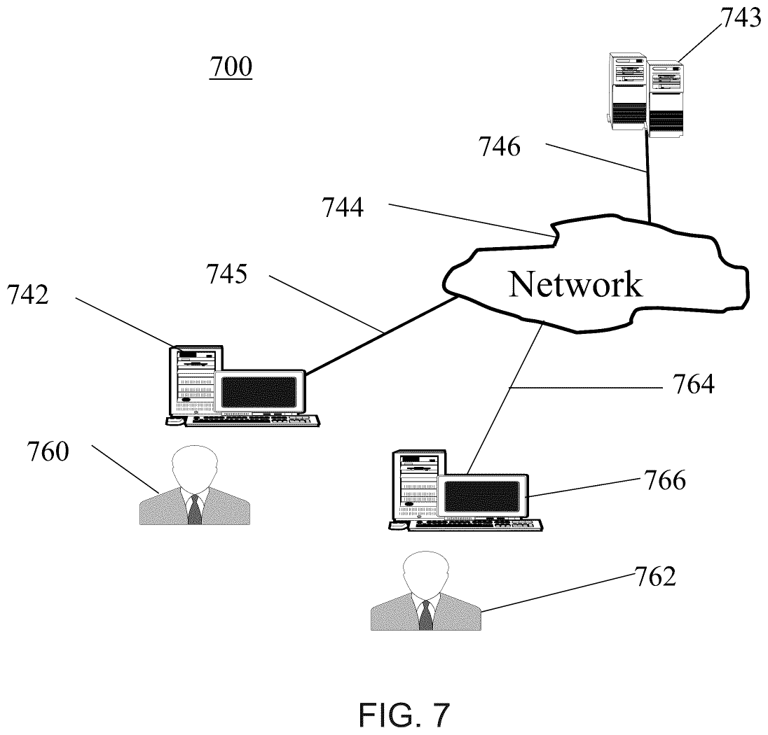

FIG. 7 is a block diagram of various example system components, in accordance with an aspect. FIG. 7 shows a communication system 700 usable in accordance with various aspects described herein. The communication system 700 includes one or more accessors 760, 762 (also referred to interchangeably herein as one or more “users”) and one or more terminals 742, 766. For example, terminals 742, 766 may include irrigation system or a related system, and/or the like. In one aspect, data for use in accordance with aspects described herein is, for example, input and/or accessed by accessors 760, 762 via terminals 742, 766, such as personal computers (PCs), minicomputers, mainframe computers, microcomputers, telephonic devices, or wireless devices, such as personal digital assistants (“PDAs”) or a hand-held wireless devices coupled to a server 743, such as a PC, minicomputer, mainframe computer, microcomputer, or other device having a processor and a repository for data and/or connection to a repository for data, via, for example, a network 744, such as the Internet or an intranet, and couplings 745, 746, 764. The couplings 745, 746, 1464 include, for example, wired, wireless, or fiberoptic links. In another example variation, the method and system in accordance with aspects described herein operate in a stand-alone environment, such as on a single terminal.

For example, in one aspect of the disclosure, the result generated by the system, as described above, may be transmitted or communicated to a remote server via a wired or wireless connection. For example, the irrigation system may transmit to a remote server a quantitative number of the targeted material, a threshold comparison outcome, or any result of the lavage process. The remote server may contain the patient's Electronic Medical Record System (EMR). In another aspect of the disclosure the remote server may be accessed by a mobile device or the remote server may actively contact a mobile device. For example, the mobile device may access the remote server containing the result via a website or propriety application loaded onto the mobile device. In another example, the remote server may push or contact the mobile device, for example, via text message, e-mail or push notifications.

The aspects discussed herein can also be described and implemented in the context of computer-readable storage medium storing computer-executable instructions. Computer-readable storage media includes computer storage media and communication media. For example, flash memory drives, digital versatile discs (DVDs), compact discs (CDs), floppy disks, and tape cassettes. Computer-readable storage media can include volatile and nonvolatile, removable and non-removable media implemented in any method or technology for storage of information such as computer readable instructions, data structures, modules or other data.

It will be appreciated that various implementations of the above-disclosed and other features and functions, or alternatives or varieties thereof, can be desirably combined into many other different systems or applications. Also that various presently unforeseen or unanticipated alternatives, modifications, variations, or improvements therein can be subsequently made by those skilled in the art which are also intended to be encompassed by the following claims.

What is claimed is:

1. A method of performing lavage comprising steps of:
(a) providing an irrigation fluid to a surgical area;
(b) removing via at least one suction line the irrigation fluid and medical waste at the surgical area to generate a fluid stream in the at least one suction line;
(c) analyzing the fluid stream via a spectroscopy test to generate a result based on a predetermined criteria;
(d) generating a feedback signal based on the result, wherein the feedback signal indicates whether the lavage has achieved the predetermined criteria; and
(e) based on the feedback signal, either (1) if the predetermined criteria has not been achieved, repeating steps (a) through (d) until the lavage has achieved the predetermined criteria, or (2) if the predetermined criteria has been achieved, ending the lavage procedure.

2. The method of claim 1, wherein the spectroscopy test is configured to test for a target material contained within the fluid stream.

3. The method of claim 2, wherein the target material includes at least one of a microorganism, debris or non-endogenous protein.

4. The method of claim 1, wherein the spectroscopy test is performed in-line on the at least one suction line.

5. The method of claim 4, wherein the spectroscopy test is continuously performed on the fluid stream.

6. The method of claim 4, wherein the spectroscopy test is performed at least once and, if necessary, periodically thereafter on the fluid stream.

7. The method of claim 1, wherein the feedback signal triggers a visual indication to a user.

8. The method of claim 7, wherein the visual indication is a light source.

9. The method of claim 1, wherein the spectroscopy test is performed remotely by obtaining a sample from the fluid stream.

10. The method of claim 1, wherein the spectroscopy test is performed by at least one of an ultraviolet measurement, a multiangle light scattering, or a fluorescence emission.

11. A system for performing the steps of claim 1.
